(19) United States
(12) Patent Application Publication
(10) Pub. No.: US 2007/0087962 A1
(43) Pub. Date: Apr. 19, 2007

(54) PHARMACEUTICAL COMPOSITIONS COMPRISING CYCLOSPORINS

(75) Inventors: Walter L. Tien, Irvine, CA (US); Richard S. Graham, Irvine, CA (US); James N. Chang, Newport Beach, CA (US)

Correspondence Address: BRENT A. JOHNSON;ALLERGAN, INC., 2525 Dupont Drive, T2-7H, Irvine, CA, 92612, US

(73) Assignee: Allergan, Inc.

(21) Appl. No.: 11/470,923

(22) Filed: Sep. 7, 2006

Related U.S. Application Data
(60) Provisional application No. 60/727,684, filed on Oct. 17, 2005.

(51) Int. Cl. A61K 38/13; A61K 31/355

(52) U.S. Cl. 514/11; 514/458

(57) ABSTRACT

A liquid comprising a therapeutically effective concentration of a cyclosporin and a vitamin E tocopherol polyethylene glycol succinate, wherein said liquid is an emulsion. is disclosed herein. Methods of treating diseases or conditions using said compositions, and medicaments related thereto, are also disclosed herein.

### CROSS REFERENCE TO RELATED APPLICATIONS

This application is based on, and claims the benefit of, U.S. Provisional Application No. 60/727,684, filed Oct. 17, 2005, and which is incorporated herein by reference.

### DESCRIPTION OF THE RELATED ART

Dry eye disease is a general term for a variety of conditions characterized by abnormalities in the tear film, which affects three million people in the United States alone. Dry eye is characterized by symptoms such as a sandy-gritty feeling in the eye, burning, irritation, or a foreign-body sensation that worsens during the day. Patients suffering from dry eye disease complain of mild to severe symptoms, and those with severe symptoms may experience constant and disabling eye irritation, and develop ocular surface epithelial disease and sight-threatening sterile or microbial corneal ulceration.

Cyclosporins are a group of nonpolar cyclic oligopeptides with immunosuppressant, anti-inflammatory, and anti-parasitic properties. Cyclosporin A is a cyclosporin which is marketed in a topical ophthalmic emulsion formulation for the treatment of dry eye by Allergan, Inc. under the tradename Restasis®. The insolubility of cyclosporins in water is an ongoing problem in the formulation of these compounds.

WO0008085 discloses “a composition for oral administration comprising (i) an immunosuppressant, e.g. cyclosporin, (ii) tocopherol (Vitamin E), tocotrienol or a derivative thereof, (iii) a short chain phospholipid, and (iv) a non-ionic surfactant”, and claims that “composition of the invention can provide for good solubility of the immunosuppressant, e.g. cyclosporin, in an excipient mixture as well as good dispersibility when placed in an aqueous environment”.

U.S. Pat. No. 5,798,333 discloses “pharmaceutical compositions which enable high concentrations of a cyclosporin and are water-soluble, such that the compositions will dissolve in aqueous media without precipitation of the cyclosporin. The compositions comprise a cyclosporin dissolved in tocophersolan and a hydrophilic organic solvent, preferably propylene glycol.” The patent further discloses that “the solvent selected should be an efficient solvent for cyclosporin, and also a solvent for tocophersolan.

Preferred organic solvents meeting these criteria include but are not necessarily limited to propylene glycol and various monoalcohols, including ethanol, benzyl alcohol, hexanol, and phenethyl alcohol.

Most preferred is propylene glycol because it has low toxicity and low volatility in addition to being an efficient solvent for cyclosporin.

The amount of propylene glycol needed to provide a stable solution of cyclosporin and tocophersolan is about 1 g per g of cyclosporin. A suitable solution preconcentrate will thus consist of 1 part cyclosporin, 7.5 parts tocophersolan and 1 part propylene glycol. “

U.S. Patent Application Publication No. 20030108626, published on Jun. 12, 2003, and filed on Nov. 1, 2001, discloses “a method and composition for treating a dry eye condition by topically applying to the eye surfaces an emulsion. . . . Includable in the mixture is a non-soluble therapeutic agent, such as cyclosporin which is effective against an eye disease and is delivered to the eye by the film”.

### DETAILED DESCRIPTION OF THE INVENTION

A liquid is disclosed herein comprising a therapeutically effective concentration of a cyclosporin and a vitamin E tocopherol polyethylene glycol succinate, wherein said liquid is an emulsion.

Another embodiment is a liquid comprising a therapeutically effective concentration of a cyclosporin, an oil, and a vitamin E tocopherol polyethylene glycol succinate, wherein said liquid is an emulsion which is ophthalmically acceptable.

Methods of treating diseases or conditions using said compositions, and medicaments related thereto, are also disclosed herein.

The compositions disclosed herein are aqueous liquid solutions according to the meaning generally understood in the art.

The term “cyclosporin” refers to any cyclosporin compounds known in the art including cyclosporin A, cyclosporin B, cyclosporin C, cyclosporin D, and cyclosporin G. In certain compositions, the cyclosporin is cyclosporin A.

The term “vitamin E tocopherol polyethylene glycol succinate” refers to an ester compound or a mixture of compounds derived from succinic acid, polyethylene glycol, and tocopherol. The compounds are diesters of succinic acid, where the two ester linkages occur to a phenolic hydroxyl group of the tocopherol and a hydroxyl group of polyethylene glycol. Polyethylene glycol is HO(CH₂CH₂O)ₙH, otherwise known as polyethylene oxide. The term tocopherol refers to a naturally occurring form of vitamin E, and may refer to a single compound or a mixture. Examples of tocopherols include α-tocopherol, dl-α-tocopherol, β-tocopherol, γ-tocopherol, and δ-tocopherol. Polyethylene glycol is the well known polymer of ethylene glycol. One useful tocopherol which is conveniently obtained commercially is sold by Eastman Chemical as Vitamin E TPGS NF, or Vitamin E TPGS 1000 which has a molecular weight of about 1,513. The US Pharmacopeia has designated tocophersolan as the name for Vitamin E TPGS NF.

The term “hydrophilic organic solvent” refers to an organic compound which is an efficient solvent for cyclosporin, and also a solvent for tocophersolan. Examples of hydrophilic organic solvents include propylene glycol and water-soluble monoalcohols, including ethanol, benzyl alcohol, hexanol, and phenethyl alcohol. In certain compositions, no hydrophilic organic solvent is present at a mass concentration greater than or equal to that of the cyclosporin. In other words, there is a greater mass of the cyclosporin than any hydrophilic solvent which may be present in the solution. In other compositions, no hydrophilic organic solvent is present at a concentration greater than half of that of the cyclosporin.

Certain compositions contain essentially no hydrophilic organic solvent.

A therapeutically effective concentration of cyclosporin is a concentration useful to observe a therapeutic effect as compared to a placebo composition having the same composition sans cyclosporin, and can be determined by a person of ordinary skill in the art without undue experimentation. In one embodiment the cyclosporin concentration is 0.001% or greater. In other embodiments, the concentration of cyclosporin is greater than 0.01%. In other embodiments, the concentration of cyclosporin is greater than 0.02%. In other embodiments, the concentration of cyclosporin is at least 0.05%. For the treatment of dry eye disease, a cyclosporin concentration of less than or equal to 1% is often adequate. In other words, in certain compositions, the concentration of the cyclosporin is at or below 1%. In other embodiments, the concentration of cyclosporin is at or below 0.2%. In other embodiments, the concentration of cyclosporin is at or below 0.15%. In other embodiments, the concentration of cyclosporin is at or below 2%. In other embodiments, the concentration of cyclosporin is about 0.05%. In other embodiments, the concentration of cyclosporin is about 0.1%.

While not intending to limit the scope of the invention in any way, vitamin E tocopherol polyethylene glycol succinate is useful as a surfactant or an emulsifier. It is useful for both for stabilizing an emulsion, as well as improving the solubility of cyclosporin in water. Thus, an effective amount of vitamin E tocopherol polyethylene glycol succinate is the amount useful to obtain the desired properties of the emulsion, obtain the desired solubility of cyclosporin, or carry out any function typical of a surfactant or an emulsifier to any extent which is discernible. Thus, an effective amount of vitamin E tocopherol polyethylene glycol succinate will depend upon the amount and kind of cyclosporin used, the properties desired, as well as what other excipients may be present in the composition. While not intending to limit the scope of the invention in any way, in many cases a vitamin E tocopherol polyethylene glycol succinate concentration of at least 0.05% is useful. In other embodiments, the concentration of vitamin E tocopherol polyethylene glycol succinate is at least 0.5%. In other cases a vitamin E tocopherol polyethylene glycol succinate concentration of at least 1% is useful. In certain cases, the vitamin E tocopherol polyethylene glycol succinate concentration may be less than or equal to 5%. In other embodiments, the concentration of vitamin E tocopherol polyethylene glycol succinate concentration is up to 20%. An oil is a hydrophobic and lipophilic liquid. In other words, it dissolves lipophilic materials, but is substantially insoluble in water. The term oil as applied herein means a single oil or a blend thereof unless otherwise indicated. There are a number of different oils which are suitable for preparing the emulsions disclosed herein. These are known to those of ordinary skill in the art.

While not a necessary consideration, the specific gravity may be important to the stability of the emulsion. Certain embodiments use an oil having a specific gravity of from about 0.9 to about 1.05. Other embodiments use an oil having a specific gravity of from about 0.95 to about 1.05. Other embodiments us an oil having a specific gravity of about 1. A combination of oils may be used to tune the specific gravity as desired.

Oils having a specific gravity of from 0.95 to 1.05 include anise oil, castor oil, clove oil, cassia oil, cinnamon oil, and the like. Oils having a specific gravity of from 0.90 to 0.95 include almond oil, corn oil, arachis oil, cottonseed oil, safflower oil, maize oil, linseed oil, rapeseed oil, soybean oil, olive oil, caraway oil, rosemary oil, peanut oil, peppermint oil, sunflower oil, eucalpytus oil, sesame oil, and the like.

One embodiment comprises an oil having a specific gravity from 0.95 to 1.05.

Another embodiment comprises Anise oil.

Another embodiment comprises Castor oil.

Another embodiment comprises Clove oil.

Another embodiment comprises Cassia oil.

Another embodiment comprises Cinnamon oil.

Another embodiment comprises an oil having a specific gravity between 0.90 and 0.95.

Another embodiment comprises Almond oil.

Another embodiment comprises Corn oil.

Another embodiment comprises Arachis oil.

Another embodiment comprises Cottonseed oil.

Another embodiment comprises Safflower oil.

Another embodiment comprises Maize oil.

Another embodiment comprises Linseed oil.

Another embodiment comprises Rapeseed oil.

Another embodiment comprises Soybean oil.

Another embodiment comprises Olive oil.

Another embodiment comprises Caraway oil.

Another embodiment comprises Rosemary oil.

Another embodiment comprises Peanut oil.

Another embodiment comprises Peppermint oil.

Another embodiment comprises Sunflower oil.

Another embodiment comprises Eucalpytus oil.

Another embodiment comprises Sesame oil.

Another embodiment comprises an oil having a specific gravity specific gravity below 0.9.

Another embodiment comprises Mineral oil.

Another embodiment comprises Coriander oil.

Another embodiment comprises Lavender oil.

Another embodiment comprises Citronella oil.

Another embodiment comprises Juniper oil.

Another embodiment comprises Lemon oil.

Another embodiment comprises Orange oil.

Another embodiment comprises Clary sage oil.

Another embodiment comprises Nutmeg oil.

Another embodiment comprises Tea tree oil.

Additional surfactants may be used in the compositions disclosed herein. While not intending to limit the scope of the invention in any way, one type of useful surfactant is a sorbitan ester. Examples include, but are not limited to, Polysorbate 20, Polysorbate 40, Polysorbate 60, and Polysorbate 80.

While not intending to limit the scope of the invention in any way, another type of useful surfactant is a stearate. Examples include, but are not limited to, glyceryl stearate, isopropyl stearate, polyoxyl stearate, propylene glycol stearate, and sucrose stearate.

While not intending to limit the scope of the invention in any way, another useful surfactant is polyethylene glycol.

While not intending to limit the scope of the invention in any way. Other useful surfactants comprise polyethylene oxide or polypropylene oxide. Examples, include, but are not limited to, polyethylene oxides, polypropylene oxides, polyethylene oxide, polypropylene oxide copolymers, alcohol ethoxylates, and alkylphenol ethoxylates.

While not intending to limit the scope of the invention in any way, another useful type of surfactant is alkyl glycosides.

While not intending to limit the scope of the invention in any way, another useful type of surfactant is alkyl polyglycosides While not intending to limit the scope of the invention in any way, another useful type of surfactant is fatty alcohols.

While not intending to limit the scope of the invention in any way, another useful type of surfactant is cellulose derivatives, including, but not limited to, hydroxypropylmethyl cellulose (HPMC) and carboxymethyl cellulose (CMC).

While not intending to limit the scope of the invention in any way, another useful type of surfactant is polyacrylic acids, including, but not limited to, Carbomers.

While not intending to limit the scope of the invention in any way, another useful type of surfactant is phospholipids, including, but not limited to, phosphatidyl chlorine and phosphatidyl serine.A liquid which is intended for ophthalmic use or ophthalmically acceptable is formulated such that it can be administered topically to the eye. The comfort should be maximized as much as possible, although sometimes formulation considerations may necessitate less than optimal comfort. In the case that comfort cannot be maximized, the liquid should be formulated such that the liquid is tolerable to the patient for topical ophthalmic use. Additionally, an ophthalmically acceptable liquid may be packaged for single use, or contain a preservative to prevent contamination over multiple uses.

As is known in the art, buffers are commonly used to adjust the pH to a desirable range for ophthalmic use. Generally, a pH of around 5-8 is desired, however, this may need to be adjusted due to considerations such as the stability or solubility of the therapeutically active agent or other excipients. Many buffers including salts of inorganic acids such as phosphate, borate, and sulfate are known.

Another commonly used excipient in ophthalmic compositions is a viscosity-enhancing, or a thickening agent. Thickening agents may be used for a variety of reasons, ranging from improving the form of the formulation for convenient administration to improving the contact with the eye to improve bioavailability. The thickening agent may comprise a polymer containing hydrophilic groups such as monosaccharides, polysaccharides, ethylene oxide groups, hydroxyl groups, carboxylic acids or other charged functional groups. While not intending to limit the scope of the invention, some examples of useful thickening agents are sodium carboxymethylcellulose, hydroxypropylmethylcellulose, povidone, polyvinyl alcohol, and polyethylene glycol.

In ophthalmic solutions, tonicity agents may be used to adjust the composition of the formulation to the desired isotonic range. Tonicity agents are well known in the art and some examples include glycerin, mannitol, sorbitol, sodium chloride, and other electrolytes.

Preservatives may be used to prevent bacterial contamination in multiple-use ophthalmic preparations. Preservatives are well known in the art, and, while not intending to be limiting, examples include polyhexamethylenebiguanidine (PHMB), benzalkonium chloride (BAK), stabilized oxychloro complexes (otherwise known as Purite®, phenylmercuric acetate, chlorobutanol, benzyl alcohol, parabens, and thimerosal are examples of useful preservatives.

In ophthalmic compositions, a chelating agent may be used to enhance preservative effectiveness. Suitable chelating agents are those known in the art, and, while not intending to be limiting, edetate (EDTA) salts like edetate disodium, edetate calcium disodium, edetate sodium, edetate trisodium, and edetate dipotassium are examples of useful chelating agents.

The compositions disclosed herein are useful in the treatment of dry eye disease, and in the preparation of medicaments for the treatment of dry eye disease. However, certain compositions disclosed herein are also useful for the treatment or prevention of other conditions or diseases which are related to immune response, inflammatory response, or parasitic or other infection.

The compositions disclosed herein are also useful for parenteral administration of a cyclosporin. A composition which is formulated for parenteral use is a composition which is formulated with the intention of administering the composition parenterally. Parenteral administration is generally characterized by injection, either subcutaneously, intramuscularly or intravenously. While not intending to limit the scope of the invention in any way, in addition to vitamin E tocopherol polyethylene glycol succinate, suitable excipients are, for example, saline, dextrose, buffering agents, and the like.

The best mode of making and using the present invention are described in the following examples. These examples are given only to provide direction and guidance in how to make and use the invention, and are not intended to limit the scope of the invention in any way.

### EXAMPLE 1

Formulations 1-4 in Table 1 below were prepared according to the following procedures.

Formulation 1 was prepared by adding 1 mg of cyclosporin into 100 μL of a 10% tocophersolan stock solution and then mixed until dissolved. To this clear solution is slowly added 900 μL of water to yield a clear solution containing 0.1% cyclosporin and 1% tocopehersolan.

Formulation 2 was prepared by adding 1 mg of cyclosporin into 10 μL polysorbate 80 and 10 μL of propylene glycol, and then mixed until dissolved. To this clear solution is slowly added 980 μL of water to yield a turbid solution containing 0.1% cyclosporin and 1% polysorabte 80 and 1% propylene glycol.

Formulation 3 was prepared by adding 1 mg of cyclosporin into 100 μL of a 10% polyoxy-40-stearate stock solution and then mixed until dissolved. To this clear solution is slowly added 890 μL of water to yield a turbid solution containing 0.1% cyclosporin and 1% polyoxy-40-stearate. This cloudy solution remained turbid even with the addition of 10 μL of propylene glycol. However, eventually the mixture became clear after standing for several days.

Formulation 4 was prepared by adding 1 mg of cyclosporin into 10 μL polyethylene glycol 400 (PEG 400) and 10 μL of propylene glycol, and then mixed until dissolved. To this clear solution is slowly added 980 μL of water to yield a turbid solution containing 0.1% cyclosporin and 1% PEG 400 and 1% propylene glycol.

TABLE 1

| | Formulation | | | |
|---|---|---|---|---|
| | 1 | 2 | 3 | 4 |
| CsA Concentration (% w/v) | 0.1 | 0.1 | 0.1 | 0.1 |
| Vitamin E TPGS (% w/v) | 1.0 | | | |
| Polysorbate 80 (% w/v) | | 1.0 | | |
| Propylene Glycol (% w/v) | | 1.0 | 1.0 | 1.0 |
| Polyoxyl-40-Sterate (% w/v) | | | | 1.0 |
| Polyethylene Glycol 400 (% w/v) | | | | 1.0 |
| Physical Appearance | Clear | PPT | PPT* | PPT |

*Mixture becomes clear after standing for several days.

While not intending to limit the scope of the invention in any way, or to be bound in any way by theory, Formulation 1, which uses a vitamin E tocopherol polyethylene glycol succinate quickly provides a clear solution, while the other formulations do not. In contrast to the formulation 1, the other formulations required propylene glycol as indicated in the procedures above. While not intending to limit the scope of the invention in any way, or to be bound by theory, the fact that vitamin E tocopherol polyethylene glycol succinate dissolves cyclosporin A also allows emulsions to be prepared with greater flexibility and ease.

In addition to being a superior surfactant, vitamin E tocopherol polyethylene glycol succinates are generally regarded in the art to have an excellent toxicology profile, and be generally less irritating than most other surfactants.

### EXAMPLE 2

The emulsion of the table below was prepared according to the following procedure.

Part I

1. Add 0.5 gm of CsA in 37.5 ml of 10% TPGS stock solution

2. Mix about 20 minutes until most of CsA in the TPGS solution

3. Add 1 gm of Castor oil in the above solution and mix another 15 minutes.

4. Add 50 ml of 5% CMC stock solution in the above solution and mix about 20 minutes.

Part II

1. In ˜390 ml of water, add 1.0 gm of Polysorbate 80 and mix until dissolved.

2. Add 5.0 gm of Glycerine and mix until dissolved.

3. Add 3.0 gm of Boric Acid and mix until dissolved.

Mix Part I and Part II for about 30 minutes, check pH and adjust pH to pH 7.3 (use ˜3 ml of 1.0 N NaOH). Heat the solution to 60-65C with mixing in a close system to prevent water loss. Homogenize this solution at 12,000 rpm at 60-65C for 15 minute and cool down to room temperature. Add 2.26 gm of purite (2.21%) to the emulsion and mix well, QS the solution to 500 ml and mix well. Sterile filter the emulsion with a 0.22 um filter.

| INGREDIENT | AMOUNT |
|---|---|
| Cyclosporin a (w/v %) | 0.1 |
| Castor oil (w/v %) | 0.20 |
| Tocophersolan - tpgs (w/v %) | 0.75 |
| Polysorbate 80 (w/v %) | 0.20 |
| Glycerin (w/v %) | 1.00 |
| Boric acid (w/v %) | 0.60 |
| Cmc—Carboxymethyl cellulose (w/v %) | 0.5(L) |
| Purite (ppm) | 100.0 ppm |
| Purified water | QS |
| SODIUM HYDROXIDE | pH adjustment |
| pH | pH = 7.3-7.5 |

### EXAMPLE 3

Dry eye is treated using the composition of Example 2. Relief of symptoms is experienced.

What is claimed is:

1. A liquid comprising a therapeutically effective concentration of a cyclosporin, an oil, and a vitamin E tocopherol polyethylene glycol succinate, wherein said liquid is an emulsion.

2. The liquid of claim 1 which contains essentially no hydrophilic organic solvent.

3. The liquid of claim 1 wherein the vitamin E tocopherol polyethylene glycol succinate is present at a concentration which is no less than 0.05%, and wherein the vitamin E tocopherol polyethylene glycol succinate is present at a concentration that is no greater than 20%.

4. The liquid of claim 1 wherein vitamin E tocopherol polyethylene glycol succinate is present at a concentration that is no less than 0.5%, and wherein the vitamin E tocopherol polyethylene glycol succinate is present at a concentration that is no greater than 5%.

5. The liquid of claim 4 comprising about 0. 1% cyclosporin A and about 0.75% vitamin E tocopherol polyethylene glycol succinate.

6. The liquid of claim 2 comprising cyclosporin A, wherein cyclosporin A is present at a concentration of at least 0.01%, and wherein cyclosporin A is not present at a concentration which is greater than 2%.

7. The liquid of claim 6 comprising cyclosporin A, wherein cyclosporin A is present at a concentration of at least 0.01%, and wherein cyclosporin A is not present at a concentration which is greater than 0.2%.

8. The liquid of claim 1 consisting essentially of a therapeutically effective concentration of cyclosporin A, an effective amount of a vitamin E tocopherol polyethylene glycol succinate, one or more oils, water, and an effective amount of one or any combination of excipients selected from the group consisting of buffers, thickening agents, tonicity agents, preservatives, and chelating agents.

9. A composition comprising a therapeutically effective concentration of cyclosporin A and an effective amount of a vitamin E tocopherol polyethylene glycol succinate, wherein said composition is an emulsion which is intended for ophthalmic use.

10. The composition of claim 1 wherein cyclosporin A is present at a concentration at or below 1%.

11. The composition of claim 10 wherein cyclosporin A is present at a concentration which is at least 0.02% and wherein cyclosporin A is present at a concentration which is less than or equal to 0.15%.

12. The composition of claim 11 comprising about 0.05% cyclosporin A.

13. The composition of claim 11 comprising about 0.1% cyclosporin A.

14. The composition of claim 13 comprising about 0.1% cyclosporin A and about 1% vitamin E tocopherol polyethylene glycol succinate.

15. A method of treating dry eye disease comprising administering to a patient an effective amount of an emulsion comprising cyclosporin A and an effective amount of a vitamin E tocopherol polyethylene glycol succinate.

16. The method of claim 15 comprising at least 0.001% cyclosporin A and wherein cyclosporin A is present at a concentration which is less than or equal to 1%.

17. The method of claim 15 wherein said solution comprises about 0.1% cyclosporin A and about 1% vitamin E tocopherol polyethylene glycol succinate.

18. The liquid of claim 1 which is intended for parenteral use.

19. The liquid of claim 1 which is intended for ophthalmic use.

20. The liquid of claim 20 comprising about 0.1% cyclosporin A and about 1% vitamin E tocopherol polyethylene glycol succinate.
